(19) United States
(12) Patent Application Publication
(10) Pub. No.: US 2005/0070789 A1
(43) Pub. Date: Mar. 31, 2005

(54) System and method for guiding surgical tools during surgical procedures

(76) Inventor: Joseph Aferzon, New Britain, CT (US)

Correspondence Address: SOFER & HAROUN LLP., 317 MADISON AVENUE, SUITE 910, NEW YORK, NY, 10017, US

(21) Appl. No.: 10/606,571

(22) Filed: Jun. 26, 2003

Related U.S. Application Data
(60) Provisional application No. 60/391,216, filed on Jun. 26, 2002.

(51) Int. Cl. A61B 5/05

(52) U.S. Cl. 600/424

(57) ABSTRACT

The present invention is directed to a method for positioning and tracing of a guiding channel during surgery, employing an imaging system. The method includes acquiring an image of a portion of a human body where surgery is to take place and fastening a surgical tool to a moving arm in at least two reference locations of the surgical tool. The surgical tool is moved, outside of the human body and near the portion where surgery is to take place. The coordinates of the two reference locations of the surgical tool are estimated in response to the movement of the surgical tool. The trajectory of the surgical device is then calculated for a distance extending from the surgical device to inside the human body. An image of the trajectory is superimposed on the acquired image, so as to allow visualization of a path extending from the surgical tool to inside the human body. The superimposed image is then displayed for visualization by the surgeon.

### RELATED APPLICATIONS

This application claims priority to a U.S. provisional application Ser. No. 60/391,216 filed on Jun. 26, 2002 and incorporated herein by reference.

### FIELD OF THE INVENTION

This invention relates to medical imaging, and more specifically to a system and method for positioning and tracing of a guiding channel during surgery on a patient.

### BACKGROUND OF THE INVENTION

In various types of surgery, there may be occasions were a surgeon is required to place instruments in precise locations inside the body. For example, numerous surgical procedures on human spine require precise placement of fixation devices into small anatomical structures. Misplaced devices frequently result in pain or neurological injury.

An example of such surgical procedure is rod installation for stabilizing bone structures such as those in the spinal column. For example, this procedure is employed to stabilize the location of bony structures in the spine relative to each other. As such, two or more anchor fasteners or screws are driven into the bony structures, such as the pedicles. A bridging rod is then inserted and attached to the screws to form a securing means to hold the corresponding bone components stable.

Recent surgical techniques strive to employ minimally invasive procedures to avoid damage to tissues intervening the skin and the spinal bone structure. To this end, screws are inserted percutaneously in first and second pedicles via guiding tubes and guidewires. The surgeon with the help of x-ray imaging tries to estimate the correct path underneath the skin for inserting the guidewire and screws.

These procedures on the spine require precise placement of the screws or fixation devices into small anatomical structures. As mentioned before, an inaccurate placement of the surgical devices can result in pain or neuroligical injury. As such surgeons use anatomical landmarks supplemented by inspection of X-ray images to guide and confirm correct placement.

However, even with the help of x-ray imaging, it is very difficult to find the correct entry axis into the appropriate bone structures, such as pedicles, requiring the surgeon to have substantial skill and experience to accomplish the task.

Thus, there is a need for a system and method that allows accurate insertion of surgical tools in the body during minimally invasive surgery.

### SUMMARY OF THE INVENTION

A system and method for positioning and tracing of a guiding channel during surgery comprises an arrangement that enables tracing of a lockable guiding channel. The system provides real time X-ray images that display the position and orientation of the guiding channel within the image scene. This allows the surgeon to visualize the guiding channel within the human body, before actually inserting a surgical tool. The system integrates the information obtained from the X-ray images with geometric calculations of the path of the guiding channel within the body.

In accordance with one embodiment of the invention, a first and second reference points on the guiding channel are configured such that their movement is restricted to a corresponding first and second fixed planes. Each of the fixed planes are defined, for example, by a moving arm that allows the movement of the reference points in all directions on the fixed plane. The system then tracks the movement of the reference points and estimates their coordinates. Based on the coordinates of the reference points, the system then draws a line on the image, extending the two points to the intended location inside the body. This allows the surgeon to visualize the direction and the path of the surgical tool inside the patient's body, based on the position of the guiding channel outside the body.

In accordance with one embodiment of the invention, the first and second fixed planes are defined by a pantograph having a fixed axis of rotation. An angle decoder measures the angle between the arms of the pantograph, referred to as rotation and deformation angles.

A computer then displays multiple anatomical images of the intended area of surgery, and superimposes the trajectory of the guiding channel on the images for visualization.

In yet another embodiment of the invention, the guiding channel is moved by a motorized mechanism so as to allow a surgeon to perform the operation remotely.

### BRIEF DESCRIPTION OF THE DRAWINGS

The present invention will be further understood from the following description with reference to the accompanying drawings, in which:

FIG. 1a illustrates an exemplary surgical step requiring accurate insertion of a surgical tool in accordance with one embodiment of the present invention.

FIG. 1b illustrates a blown-up view of a vertebral body, wherein a surgical tool has been inserted into a pedicle.

FIG. 2 illustrates a fluoroscopy system configured to obtain X-ray images of a patient in accordance with one embodiment of the present invention.

FIG. 3a illustrates an arrangement for a moving guiding channel via guiding arms in accordance with one embodiment of the present invention.

FIG. 3b illustrates the operation of the angle encoders using a pantograph mechanism in accordance with one embodiment of the present invention.

FIG. 4a illustrates a pantoguide employing angle encoders in accordance with one embodiment of the present invention.

FIG. 4b illustrates a holding mechanism to moveably secure a guiding channel in accordance with one embodiment of the present invention.

FIG. 5 illustrates the path of X-ray beams emitted from a source at location F to an operating space and to an image space in accordance with one embodiment of the invention.

FIG. 6 illustrates an exemplary X-ray image with two guiding arms and four fiducials in accordance with one embodiment of the invention.

FIG. 7 is a functional block diagram illustrating the operation of the surgery system in accordance with one embodiment of the present invention.

FIG. 8 illustrates the operation of the guiding arms operated by a motor in accordance with one embodiment of the invention.

### DETAILED DESCRIPTION OF THE DRAWINGS

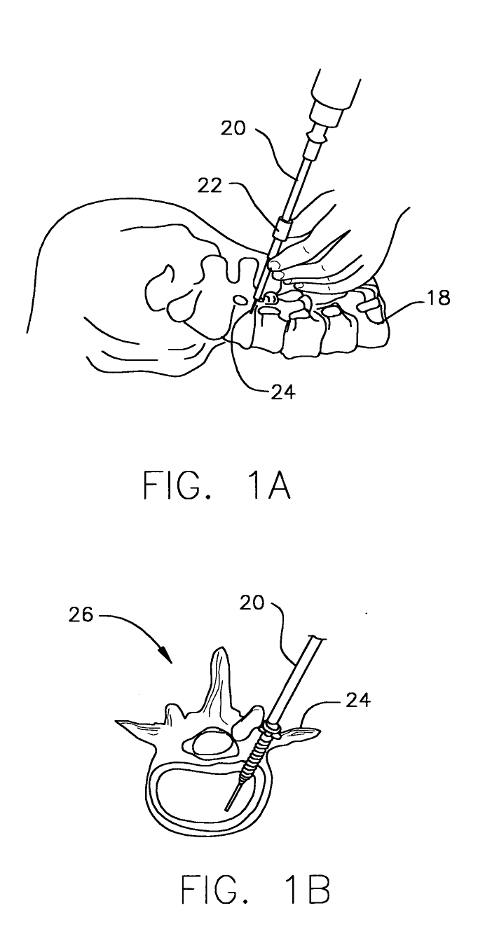

FIG. 1a illustrates an example of a surgical step for inserting into a human spine 18, a surgical tool, such as a guidewire 20, through a dilation sleeve 22. As mentioned above, the exemplary purpose for this procedure is to insert anchor screws into pedicle 24 of a patient. FIG. 1b illustrates a blown-up view of a vertebral body 26, wherein guidewire 20 has been inserted into pedicle 24.

During this procedure, the surgeon obtains numerous fluoroscopy images to verify the trajectory of the guidewire, and its final position. Thereafter, anchor screws are inserted via the guidewire.

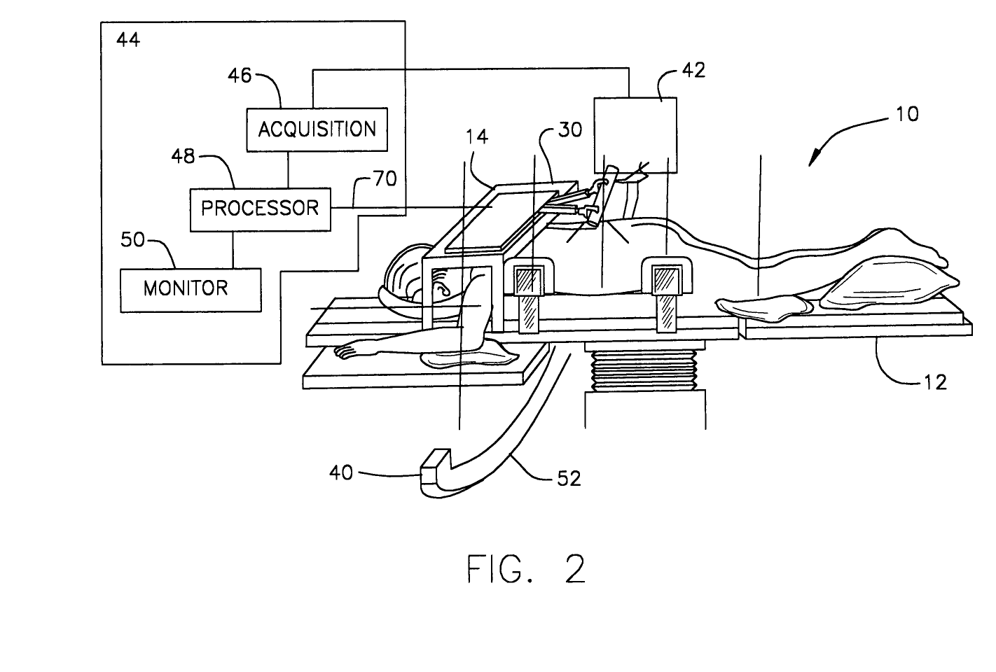

In order to minimize the amount of fluoroscopy and improve the accuracy of the procedure the present invention relates to a procedure for superimposing the trajectory of a guiding channel on an image obtained by fluoroscopy. As such FIG. 2 illustrates one embodiment of the present invention, wherein a fluoroscopy system 44 is configured to obtain X-ray images of a patient 10.

Patient 10 lies face down on an operation table 12. A guiding arm support table 14 is placed over the patient's back. Table 14, in accordance with one embodiment of the invention, is placed on the operation table, while the patient's hands extend through the legs of the table. Guiding arms 16 and 16′ are attached to a guiding arm drive unit 30. The guiding arms are positioned parallel to each other with a fixed distance h apart. Each driving arm moves on a fixed plane parallel to the plane of table 14.

A guiding channel 8 is coupled to the two guiding arms. As will be described in more detail below, the guiding arms allow guiding channel 8 to move in any desired direction over the patient's body. It is noted that although the embodiment of the invention described above employs a guiding channel 8, the invention is not limited in scope in that respect. For example, any other surgical tool can be employed by attaching the surgical tool to the guiding arms, as described in reference with the example of the guiding channel.

Once a desired location is achieved, the guiding arms are locked by a locking mechanism, so that the position of the guiding channel remains fixed for the remainder of the surgery. Thereafter, various surgical tools are inserted into the patient's back through the guiding channel. Although the exemplary embodiment described in reference with FIG. 2 relates to a surgical procedure on the back, the invention is not limited in scope in that respect, and any surgical procedure that requires an accurate localization of entry into the body is contemplated within the present invention.

Fluoroscopy processing system 44 is coupled to X-ray source 40 and a camera 42. Source 40 is configured to emit pulses at the patient's body. Camera 42 is configured to receive the X-ray pulses as they have passed through the body, so as to provide an image of the internal parts of the patient. In accordance with various embodiments of the invention, camera 42 may be a CRT (cathode ray tube) camera, or a CCD (charged coupled device) camera. Typically, source 40 and camera 42 are coupled via a C-arm arrangement. The C-arm allows them to rotate around the patient's body in a fixed relation with each other.

Camera 42 is coupled to the fluoroscopy processing system 44. System 44 includes a data acquisition unit 46, which is configured to receive the data from camera 42 and store the images in an image matrix for later viewing on a monitor such as monitor 50. A processor 48 is configured to control the operation of system 44 and perform geometrical calculations so as to superimpose on the displayed images, the trajectory of guiding channel 8 extended into the patient's body.

Thus, during operation, a surgeon obtains multiple images from the vicinity of the section of the body, wherein operation will be performed, referred to as the surgical field. The X-ray source may emit pulses at various specified orientations of C-arm 52, which according to the surgeon can provide images best identifying the relevant anatomy of the patient. Thereafter, guiding channel 8 is manipulated through guiding arms 30. The coordinates of the reference points on the guiding channel are then estimated by processor 48, so as to superimpose the trajectory of the guiding channel on the images previously obtained by the surgeon. Once the optimal trajectory is found, guiding channel 8 is fixed and the remaining steps of the surgery, including the steps of inserting various surgical tools in to the intended area of the body through the guiding channel is accomplished.

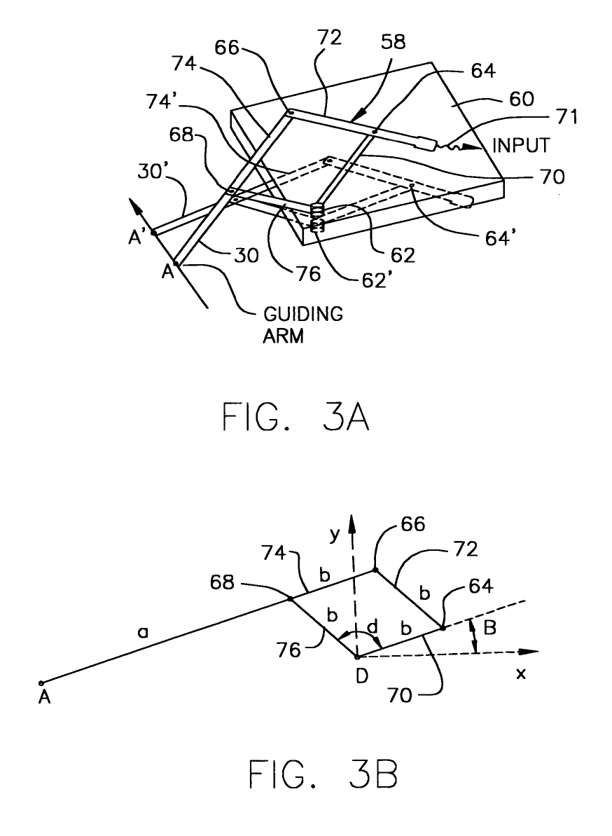

FIG. 3a illustrates an arrangement for moving guiding channel 8 via guiding arms 30 in accordance with one embodiment of the present invention, although the invention is not limited in scope in that respect. For example, other mechanisms for moving guiding channel 8 and estimating the coordinates of the movement of reference points on the guiding channel are also contemplated, in accordance with various embodiments of the invention.

Thus, FIG. 3a illustrates a pantograph 58, having four arms 70, 72, 74 and 76, which are moveable about pivot points 62, 64, 66 and 68. Moving arm 74 extends beyond pivot point 68 to form guiding arm 30. Guiding arm 30 is attached to guiding channel 8 at point A. Pivot point 62 is fixedly connected to a pantograph support table 60. As such the four arms of the pantograph can only move on a fixed plane tangential to support table 60.

FIG. 3a illustrates a second pantograph 58′, having four arms, which are moveable about corresponding pivot points. The second pantograph includes a moving arm 74′ that extends beyond pivot pint 68′ to form a guiding arm 30′. Guiding arm 30′ is attached to guiding channel 8 at point A′. Pivot point 62′ is fixedly connected to a corresponding pantograph support table 60′ (not shown). The four arms of the second pantograph can only move on a fixed plane tangential to support tables 60 and 60′. Thus, points A and A′ of guiding channel 8 can move in different directions allowing a plurality of orientations for optimum placement of the guiding channel.

Pantoguides 58 and 58′ include angle encoders that measure the angle deformations of the four arms as points A and A′ are manipulated. The angle measurements are then converted to electronic signals and sent via line 70 to processor 48.

FIG. 3b illustrates the operation of the angle encoders using the pantograph mechanism of a pantograph such as 58 or 58′ illustrated in FIG. 3a, in accordance with one embodiment of the present invention. In this embodiment, the four arms of the pantograph have equal length b. The plane of movement of the pantograph arms is defined by x and y coordinates.

The angle Δ between arms 70 and 76 is referred to as the deformation angle. Furthermore the angle E between arms 70 and the x axis is referred to as the rotation angle. As point A moves in one direction along the x axis, the deformation angle Δ expands and contracts accordingly. Similarly as point B moves in one direction along the y axis, the rotation angle E expands and contracts accordingly.

Thus, it is possible to estimate the coordinates of point A as follows:
Aₓ=−a−b Sin(Δ−90)
A_{y}=b Cos(Δ−90)
wherein a is the distance of guiding arm 30 from pivot point 68. Furthermore, rotation of the pivot point D about a rotation axis extending perpendicular to the plane of the paper is defined as:
   xᵣ=x Cos E−y Sin E
   yᵣ=x. Sin E+y Cos E

As such, Aₓ and A_{y} for a combination of change in deformation and rotation angles can be estimated as follows:
Aₓ=−a. Cos E−b. Sin(Δ−90). Cos E−b. Cos(Δ−90). Sin E
A_{y}=−a. Sin E−b. Sin(Δ−90). Sin E+b. Cos(Δ−90). Cos E

Although the above solution is provided for a pantograph with equal sized arms, the invention is not limited in scope in that respect. For example a rectangular pantograph with arms of the size of b in width and c in height can be employed in accordance with other embodiments of the invention.

Furthermore, for the embodiment of the invention that includes two pantographs placed in parallel to each other, such as the one disclosed in FIG. 3a, the coordinates of the second reference point A′ can also be calculated as described above. Hence processor 48 can derive the line equation passing through points A and A′ so as to superimpose the image of the line over the X-ray image originally obtained by the surgeon.

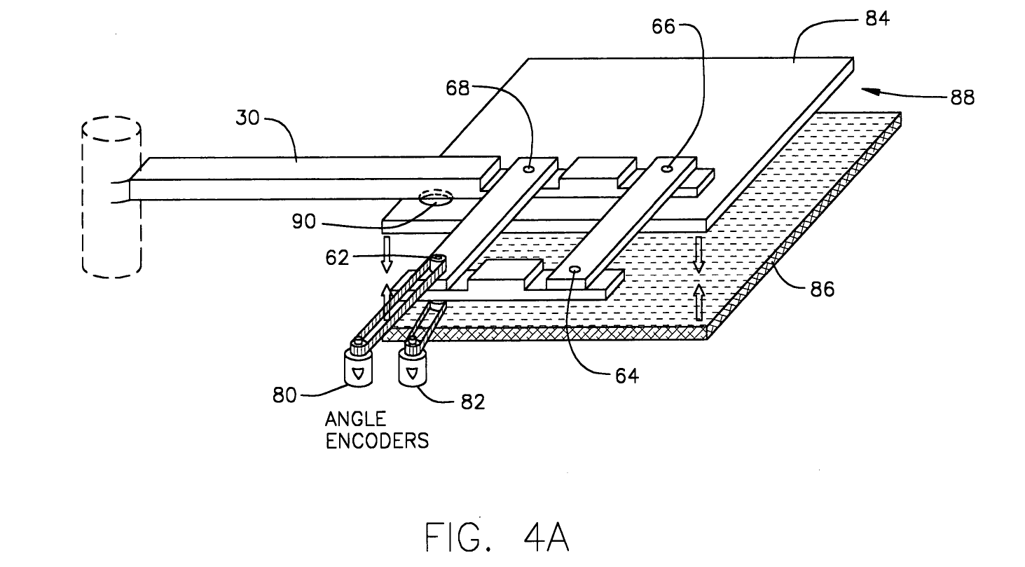

FIG. 4a illustrates a pantoguide 58 employing angle encoders 80 and 82 in accordance with one embodiment of the present invention. Thus angle encoder 80 is configured to measure deformation angle Δ, while angle encoder 82 is coupled to measure rotation angle E. It is noted that the structure and operation of angle encoders employed in the present embodiments of the invention may be selected from a variety of available encoders and as such the invention is not limited in scope in a particular structure of angle encoders. For example, mechanical, optical, electromagnetic and holographic encoders can be employed without departing from the scope of the present invention.

As illustrated in FIG. 4a pantoguide 60 is contained in an enclosure 88, having a top surface 84 and a bottom surface 86, such that guiding arm 30 extends outside of the enclosure, while the remaining components of the pantoguide are disposed within the housing.

FIG. 4b illustrates a fastening unit 96 attached to moving arm 30. Fastening unit 96 includes a rotating arm 92 and a clasping hook 94 attached to it. Clasping hook 94 attaches to guiding channel 8. As the guiding channel freely moves, rotating arm 92 rotates in the direction of movement. Once the surgeon is satisfied with the orientation of guiding channel 8, rotating arm 92 can be locked so that the orientation of the guiding channel remains fixed through out the remainder of the surgery. It is noted that the invention is not limited in scope in that respect and fastening units other than unit 96 can be used without departing from the invention.

For fluoroscopy applications, as compared to CT scanning applications, it is necessary to take into account the projection of the pulses passing through the surgery space on to the image space, such as camera 42.

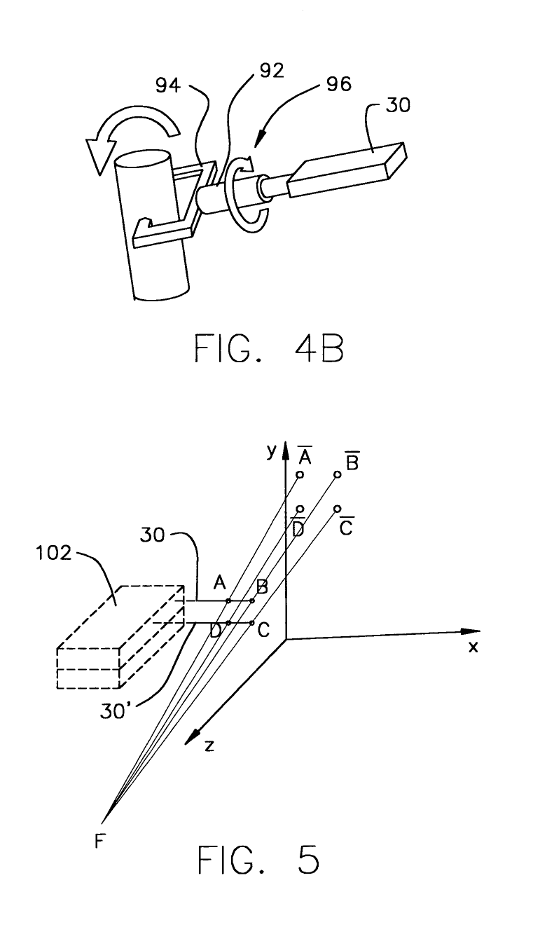

FIG. 5 illustrates the path of X-ray beams emitted from source 40 at location F. Guiding arms 30 and 30′ are extended from a support base 102. As mentioned before, in accordance with one embodiment of the invention, moving arms 30 and 30′ are respectively operated by an arrangement such as the pantoguide described and illustrated in reference with FIGS. 3 and 4.

Guiding arms 30 and 30′ include four reference markers such as A, B, C, D, referred to as fiducials. The image of these four fiducials is projected on the image plane, such as 42 defined by coordinates x, y, z, wherein z=0, to form image points \overline{A} \overline{B} \overline{C} \overline{D}.

Since the distance between fiducials A, B, C, and D are known, it is possible to determine the transform function for transforming the coordinates of image points on the image plane to find the coordinates of the fiducials. Once the transform function is determined, the system measures the coordinates of the reference points on guiding channel 8, as described in reference with FIGS. 3 and 4, and thereafter obtains the coordinates of the image of the reference points on the guiding channel within the image plane, by using the inverse of the transform function. Thereafter, system 44 superimposes a line on the image, based on the coordinates of the image of the reference points on the guiding channel.

In accordance with one embodiment of the invention, and in reference with FIG. 5, the transfer function can be derived as described hereinafter.

X-ray beam originating at F projects onto plane
˜AB˜=˜BC˜=˜CD˜=˜AD˜=S

Given projections of points A, B, C, D equal to
(\overline{A}ₓ, \overline{A}_{y}),(\overline{B}ₓ,\overline{B}_{y}),(\overline{C}ₓ,\overline{C}_{y}),(\overline{D}ₓ,\overline{D}_{y})
it is possible to find the 3-D Coordinates of
   A,B,C,D=(\overline{A}ₓ,\overline{A}_{y}, \overline{A}_{z}),(\overline{B}ₓ,\overline{B}_{y};\overline{B}_{z}),(\overline{C}ₓ;\overline{C}_{y};\overline{C}_{z}),(\overline{D}ₓ,\overline{D}_{y},\overline{D}_{z})
in space defined by +F

Thereafter, given the coordinates of A, B, C, D it is possible to find the projections of orthogonal unit vectors onto plane L,
wherein space L is such that F=(0;0, ƒ), \overline{A}=(\overline{A}ₓ, \overline{A}_{y}; 0),
\overline{B}=(\overline{B}ₓ; \overline{B}_{y}; 0), \overline{C}=(\overline{C}ₓ, \overline{C}_{y}, 0), \overline{D}=(\overline{D}ₓ, \overline{D}_{y}; 0)
Thus unit vectors AB and CB are defined as:

$$= \frac{\overline{AB}}{\vert \overline{AB} \vert} ; = \frac{\overline{CB}}{\vert \overline{CB} \vert}$$

It is noted that |AB|=distance between A & B; and <AFB is the angle between line AF≈BF
AB=vector A to B, ABoBC=dot product
Solution for A, B, C (D is similar), can be obtained as follows:

To this end, it is considered that α=<AFB=<\overline{A}F\overline{B}; β=<BFC=<\overline{B}F\overline{C}; and γ=<AFC=<\overline{A}F\overline{C}
Based on law of Cosines

$$1 ) \{ \begin{matrix}\vert FA {\vert}^{2} + \vert FB {\vert}^{2} - 2 \cdot \vert FA \vert \cdot \vert FB \vert \cdot cos α = \vert AB {\vert}^{2} = {s}^{2} & ( i ) \\ \vert FB {\vert}^{2} + \vert FC {\vert}^{2} - 2 \cdot \vert FB \vert \cdot \vert FC \vert \cdot cos β = {s}^{2} & ( ii ) \\ \vert FA {\vert}^{2} + \vert FC {\vert}^{2} - 2 \cdot \vert FA \vert \cdot \vert FC \vert \cdot cos γ = 2 {s}^{2} & ( iii )\end{matrix}$$

where cos α=F\overline{A}\hat{ }F\overline{B}; cos β=F\overline{B}\hat{ }F\overline{C}; cos γ=F\overline{A}\hat{ }F\overline{C}
To simplify: |FA|=a; |FB|=b; |FC|=c
a²+b²−2ab·cos α=a²−2ab·cos α+b²·cos²α−b²·cos²α+b²(a−b·cos α)²+b²−b² cos²α=s²

$$1 ) \{ \begin{matrix}a = \sqrt{{s}^{2} - {b}^{2} + {b}^{2} \cdot {cos}^{2} α} + b \cdot cos α & ( i ) & also \sqrt{{s}^{2} - {b}^{2} \cdot {sin}^{2} a} + b \cdot cos α \\ c = \sqrt{{s}^{2} - {b}^{2} + {b}^{2} \cdot {cos}^{2} β} + b \cdot cos & ( ii ) & \\ {a}^{2} + {c}^{2} - 2 a \cdot c \cdot cos γ - 2 {s}^{2} = 0 & ( iii ) & \end{matrix}$$

Rather than solve this equation empirically, system 44 employs an approximation algorithm as commonly known to those skilled in the art.

Thus system 44 picks numbers for b between min. & max.

Since distance has to be≧0 min=0; max has to be such that s²−b²·sin²α≧0 & s²−b²·sin²E≧0

Thus max=^{s}/sin α or max=^{s}/sin β whichever is smaller.

Thus, system 44 first takes the following step:

divide (max−min) into number of equal parts, num.
   Second, system 44 takes the following step:
Increment b from min to max by max−min/num;
Plug into (i) & (ii) & find a & c
   Third, system 44 takes the following step:
Plug a & c into result=a²+c²−2ac cos γ−2s²
   Forth, system 44 takes the following step:
Find b, which gives smallest result (b)
   At the fifth step system 44 takes the following step:
Define min=b−(max−min/num); max=b+(max−min/num)
The system then goes back to step 1 and repeats a number of times.
Thereafter, the system finds the coordinates of A; B & C in X-ray space

$$A = F + FA \cdot \frac{a}{\vert \overline{FA} \vert} ; B = F + F \overline{B} \frac{b}{\vert F \overline{B} \vert} ; C = F + F \overline{C} \frac{c}{\vert F \overline{C} \vert} .$$
& vector:

$$Ξ = \frac{AB}{\vert AB \vert} or \frac{AB}{s} u = \frac{CB}{s}$$
& w= [symbol] νu (cross product-+/−sign)33
   The center of a guiding channel, with two reference points, C_{UP} & C_{DN} is distance θ from points B & C. Then guiding channel passes through: C_{UP}=B+θ [symbol] and C_{DN}=C+θu.

As pantograph moves by (x; y) in its own plane, this movement in X-ray space is x· [symbol] ; y·w; or its new coordinates=
C_{UP}=B+θ [symbol] +x_{UP}· [symbol] +y_{UP}·w
C_{DN}=C+θ [symbol] +x_{DN}· [symbol] +y_{DN}·w
The system then obtains the superimposed image of the line by finding the equation of the line through (F & C_{UP}); (F & C_{DN}) & finding its intersection with plane. This would represent the necessary projection.

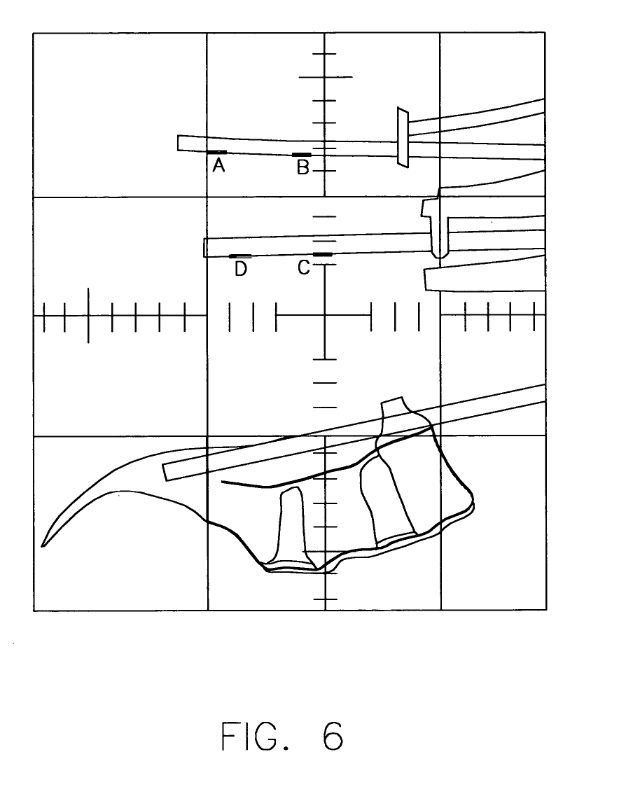

FIG. 6 illustrates an exemplary X-ray image with two guiding arms 30 and 30′ and the four fiducials as explained above in reference with FIG. 5.

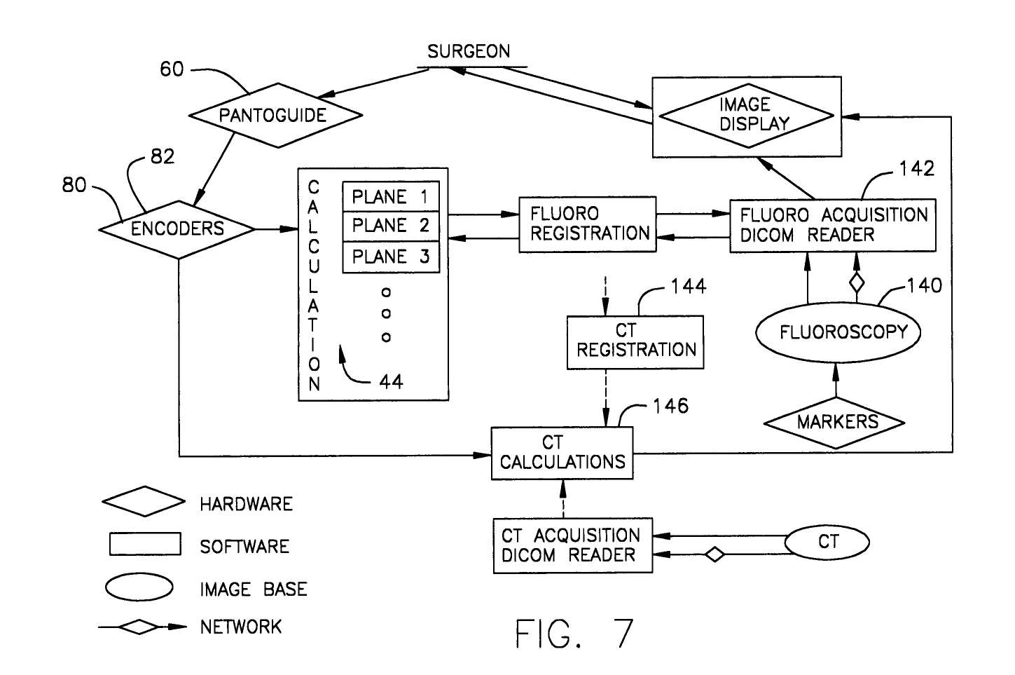

FIG. 7 is a functional block diagram illustrating the operation of the surgery system in accordance with one embodiment of the present invention. Thus, pantoguides 60 are coupled to encoders 80 and 82. The encoders provide appropriate angle measurement signals to system 44 for geometrical calculations.

Fluoroscopy system includes matrices 140 for storing images acquired by the acquisition system. These images are provided to a DICOM reader 142 for processing the data and converting them to an appropriate format for dispay on monitor 50. The processed images are also provided to fluoro registration unit and thereafter to system 44, for calculating the projection of the four markers or fiducials ABCD as explained above in reference with FIG. 5.

The system also employs a CT registration unit, which provides data to CT calculation unit 146. The operation of the CT registration unit relates to calculating projections from a surgery space to an image space of a CT device, for systems that employ a CT scan instead of a fluoroscopy imaging.

Thus, the surgeon employs the guiding arms, such as the pantoguide to find an optimum location for guiding channel 8. As the guiding arms are moving, the angle encoders provide the movement of the reference points on the guiding channel to system 44. System 44 performs calculations as described above in reference with FIGS. 3-5 in various image planes as specified by the surgeon.

Meanwhile, the surgeon examines the X-ray images displayed on a monitor such as 50. The image of fiducials are obtained via fluoroscopy processing system 44. The system performs calculations on various image planes to obtain the coordinates of the reference points of the guiding channel and project those coordinates onto each of the image planes. The trajectory of the guiding channel is then superimposed on each image to allow the surgeon the visualize the direction of the guiding channel into the human body.

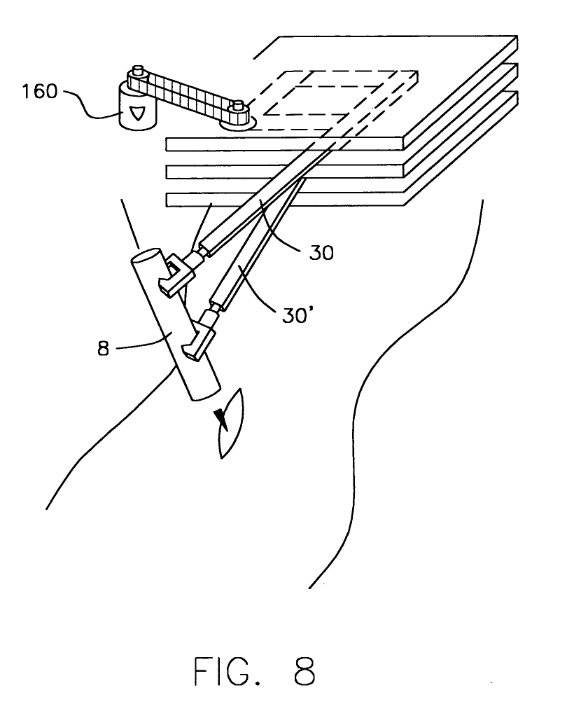

FIG. 8 illustrates the operation of the guiding arms 30 and 30′ wherein the pivot point 62 is operated by a motor 160. This configuration of the invention allows for an arrangement wherein the guiding channel can be remotely manipulated. This allows procedures to be accomplished remotely, as will be appreciated by those skilled in the art.

While only certain features of the invention have been illustrated and described herein, many modifications, substitutions, changes or equivalents will now occur to those skilled in the art. It is therefore, to be understood that this application is intended to cover all such modifications and changes that fall within the true spirit of the invention.

1. A method for positioning and tracing of a guiding channel during surgery, employing an imaging system, said method comprising the steps of:
acquiring an image of a portion of a human body where surgery is to take place;
fastening a surgical tool to a moving arm in at least two reference locations of said surgical tool;
moving said surgical tool outside of the human body and near said portion where surgery is to take place;
estimating the coordinates of said two reference locations of said surgical tool in response to said movement of said surgical tool;
calculating the trajectory of said surgical device for a distance extending from said surgical device to inside said human body;
superimposing an image of said trajectory on said acquired image, so as to allow visualization of a path extending from said surgical tool to inside said human body; and
displaying said superimposed image for visualization by said surgeon.

2. The method in accordance with claim 1 further comprising the steps of acquiring multiple images of said portion of human body before said estimating step.

3. The method in accordance with claim 2, further comprising the step of calculating said trajectory for each one of said acquired images.

4. The method in accordance with claim 3, further comprising the step of displaying said trajectory for visualization on each of said acquired images.

5. The method in accordance with claim 1 wherein said step of moving said surgical tool further comprises the step of employing a pantoguide, fixedly coupled to a plane.

6. The method in accordance with claim 5, further comprising the step of measuring a deformation angle and a rotation angle of said pantoguide as said surgical tool moves.

7. The method in accordance with claim 1 further comprising the step of estimating the projection of images of objects in the proximity of surgery area on to the image space of said acquired images.

8. The method in accordance with claim 7 further comprising the steps of estimating the projection of images of a plurality of markers attached to said moving arms, with previously known locations.

9. The method in accordance with claim 8 further comprising the step of employing a motor mechanism for moving said surgical tool remotely.

10. The method in accordance with claim 1 further comprising the step of restricting movement of said first and second reference locations to a corresponding first and second fixed planes.

11. A system for positioning and tracing of a guiding channel during surgery on a patient, said system comprising:
a first moving arm pivotally fixed to a first fixed plane;
a second moving arm pivotally fixed to a second fixed plane;
a pair of guiding channel fasteners each coupled to said first and second moving arms and configured to attach to a guiding channel at a corresponding first and second reference locations; and
an encoder unit coupled to said moving arm, said encoder unit configured to provide signals so as to allow an estimate of the coordinates of said reference locations.

12. The system in accordance with claim 11 further comprising a processor coupled to said encoder unit configured to calculate a trajectory of a line extending from said surgical tool towards the interior portion of said patient.

13. The system in accordance with claim 12 further comprising an imaging unit configured to acquire images of the interior portion of said patient's body.

14. The system in accordance with claim 13 further comprising a display means configured to display the trajectory of said guiding channel within the patient's body.

15. The system in accordance with claim 14, wherein said moving arms are each attached to a pantoguide.

16. The system in accordance with claim 15, wherein said encoder unit further comprises angle encoders for measuring deformation and rotation angle of said pantoguide.

17. The system in accordance with claim 16 further comprising a motor mechanism coupled to said moving arms so as to remotely control the movement of said moving arms.

18. A system for positioning and tracing of a guiding channel during surgery on a patient, said system comprising:
a first pantoguide unit having four moving arms pivoted together by pivot points, wherein one of said pivot points is fixedly attached to a first surface, and wherein one of said arms is extended to define a first moving arm;
a second pantoguide unit having four moving arms pivoted together by pivot points, wherein one of said pivot points is fixedly attached to a second surface, and wherein one of said arms is extended to define a second moving arm, said first and second surface are fixedly apart from each other;
a first fastening unit coupled to said first moving arm and a second fastening unit coupled to said second arm, wherein said fastening units are configured to attach to a guiding channel at a first and second reference locations; and
an angle encoder coupled to each of said first and second pantoguides configured to provide information corresponding to deformation and rotation angles of said pantoguides as said first and second reference locations move in a surgery space.

19. The system according to claim 18 wherein said first and second surfaces are disposed in parallel to each other.

20. The system according to claim 19 further comprising a processor configured to receive said information provided by said angle encoders.

21. The system in accordance with claim 20, wherein said processor is further configured to calculate the coordinates of said first and second reference locations based on said angle information received from said angle encoders.

22. The system in accordance with claim 21, wherein said processor is further configured to calculate a trajectory of a line extending from said first and second reference locations based on said calculated coordinates.

23. The system in accordance with claim 22 further comprising an image acquisition system configured to acquire images of interior portions of a patient proximate to location of an intended surgery.

24. The system in accordance with claim 23, wherein said processor is further configured to superimpose said calculated trajectory on images acquired by said image acquisition system so as to allow visualization of said line within the patient's body.

25. The system in accordance with claim 23, wherein said processor is further configured to calculate the projection of images from said surgery space to an image plane of said acquired images.

26. The system in accordance with claim 24 further comprising a motor mechanism so as to remotely control the movement of said moving arms.
